(19) United States
(12) Patent Application Publication
(10) Pub. No.: US 2025/0092540 A1
(43) Pub. Date: Mar. 20, 2025

(54) CATALYST ELECTRODE FOR PRODUCTION OF 2.5-FURANDICARBOXYLIC ACID AND METHOD FOR PREPARATION THEREOF

(71) Applicant: KOREA INSTITUTE OF SCIENCE AND TECHNOLOGY, Seoul (KR)

(72) Inventors: Dong Ki LEE, Seoul (KR); Ung LEE, Seoul (KR); Hyung-Suk OH, Seoul (KR); Byoung Koun MIN, Seoul (KR); Dahye WON, Seoul (KR); Jai Hyun KOH, Seoul (KR); Woong Hee LEE, Seoul (KR); Jongin WOO, Seoul (KR); Byeong Cheul MOON, Seoul (KR)

(21) Appl. No.: 18/413,987

(22) Filed: Jan. 16, 2024

(30) Foreign Application Priority Data
Sep. 14, 2023 (KR) 10-2023-0122541

(51) Int. Cl. C25B 11/061; C25B 3/05; C25B 3/07; C25B 11/037; C25B 11/091

(52) U.S. Cl. CPC C25B 11/061; C25B 3/05; C25B 3/07; C25B 11/037; C25B 11/091

(57) ABSTRACT

A catalyst electrode for production of 2,5-furandicarboxylic acid according to an embodiment of the present invention includes: a first metal substrate; and a second metal hydroxide coating layer located on a surface of the first metal substrate, wherein the first metal substrate includes one or more micro-sized first metal particles on the surface thereof, and the second metal hydroxide coating layer may be located on a surface of the first metal particles.

### CROSS-REFERENCE TO RELATED APPLICATION

This application claims priority to and the benefit of Korean Patent Application No. 10-2023-0122541 filed in the Korean Intellectual Property Office on Sep. 14, 2023, the entire contents of which are incorporated herein by reference.

### BACKGROUND OF THE INVENTION

### (a) Field of the Invention

Embodiments of the present invention relate to a catalyst electrode for electrochemical production of 2,5-furandicarboxylic acid and a method for preparation thereof, and more particularly, to a mixed catalyst electrode capable of synthesizing high purity FDCA using a crude HMF solution as a raw material and a method for preparation thereof.

### (b) Description of the Related Art

The life-cycle greenhouse gas (GHG) emissions from petrochemical-based plastics are assumed to be 6.5 GtCO₂e, which will be approximately 15% of the global carbon budget by 2050. This led to an urgent need to develop sustainable plastics made of carbon-neutral materials such as biomass or metabolic product of microorganisms. Among them, 2,5-furandicarboxylic acid (FDCA) is considered as the most promising biomass-derived chemical building block for a potential alternative to terephthalic acid (TPA). Indeed, polyethylene furandicarboxylate (PEF) produced by the polymerization of the FDCA and ethylene glycol showed market-ready physiochemical properties for drink and food packaging. Nevertheless, commercialized large-scale production of PEF has yet to be accomplished. The high cost of FDCA compared to TPA is one of the major drawbacks of the commercialization of PEF.

Typically, FDCA is synthesized by the acid-catalyzed dehydration of fructose, followed by the carboxylation of functional groups at both ends. The fructose dehydration results in a 5-hydroxymethylfurfural (HMF) which is an important intermediate in the conversion process of fructose to FDCA. Various strategies for the thermochemical and electrochemical conversion of HMF to FDCA have been largely reported, with excellent FDCA yields and selectivities of over 95%. However, such performance can only be achieved using highly refined HMF (purity>99%) costing at least $1000 per kg, which is a major contributor to the high-cost FDCA. This is because the crude HMF solution resulting from the fructose dehydration inevitably contains various impurities, thus that an additional purification process involving multiple solvent extraction and filtration steps is necessary to obtain high-purity HMF

To solve the problems, KR10-2021-0148900 discloses a catalyst capable of promoting a process of oxidizing 5-hydroxymethylfurfural (HMF) to 2,5-furandicarboxylic acid (FDCA), and specifically, discloses a catalyst electrode having a higher content of Ni³⁺ than Ni²⁺ in a catalytic compound located on a metal substrate. In addition, KR 10-2023-0058264 discloses a catalyst electrode applied to a process for electrochemically producing 2,5-furandicarboxylic acid, which can improve production efficiency while reducing the generation of impurities. However, it was found that it was still insufficient to solve the challenge of efficiently synthesizing FDCA while omitting the purification process of the crude HMF solution.

Meanwhile, the synthesis of FDCA from HMF requires a reaction that oxidizes alcohol and aldehyde groups of HMF into carboxylic acid without deformation of the central furan ring. In the thermochemical conversion of HMF to FDCA involving a reaction condition of high-temperature/high-pressure, not only the conversion of the alcohol/aldehyde groups of HMF, but also various side reactions such as opening of the furan ring or polymerization of reaction intermediate products easily occur. In particular, when by-products coexist with HMF, side reactions that degrade HMF are further accelerated. For this reason, the use of a highly pure HMF solution is essential to obtain high-purity FDCA through the thermochemical reaction.

An electrochemical oxidation of HMF at room temperature and pressure using transition metal catalysts can selectively convert alcohol and aldehyde groups to carboxylic acids without any reactivity to the furan ring or other functional groups. This allows the synthesis of FDCA by selectively converting HMF, even when the HMF is mixed with various compounds and side products. In other words, using an electrochemical reaction with a well-designed catalyst, high-purity FDCA may be synthesized without the purification process of the crude HMF solution, thereby significantly reducing the production cost of FDCA.

Therefore, it is necessary to develop a new electrocatalyst can selectively produce FDCA from the crude HMF solution, and a process for production of FDCA using the same.

### SUMMARY OF THE INVENTION

The present disclosure attempts to provide a new catalyst electrode capable of synthesizing high-purity FDCA using a crude HMF solution as a raw material and a process for production of FDCA using the same.

An exemplary embodiment of the present invention provides a catalyst electrode for production of FDCA including: a first metal substrate; and a second metal hydroxide coating layer located on a surface of the first metal substrate, wherein the first metal substrate includes one or more micro-sized first metal particles on the surface thereof, and the second metal hydroxide coating layer may be located on a surface of the first metal particles.

The micro-sized first metal particles may be secondary particles formed by agglomerating first metal primary particles with an average particle size (D50) of 5 μm or less.

The second metal hydroxide may be dispersed and located on the surface of the first metal substrate in an island shape.

The second metal hydroxide may be located on 5% to 20% of a surface area of the first metal substrate.

The second metal hydroxide may have an average thickness in a range of 0.1 μm to 1 μm.

The first metal substrate may include one or more types selected from a nickel foam and a nickel foil.

The second metal hydroxide coating layer may be a copper hydroxide coating layer.

Another embodiment of the present invention provides a method for preparation of a catalyst electrode for production of FDCA including: growing micro-sized first metal particles on a surface of a first metal specimen; and forming a second metal hydroxide layer on the surface of the first metal specimen on which the micro-sized first metal particles are grown, wherein the first metal and the second metal may be different transition metals.

In the growing of the micro-sized first metal particles on the surface of the first metal specimen, electrodeposition may be performed in a constant current method by immersing the first metal specimen in a first metal salt solution.

In the growing of the micro-sized first metal particles on the surface of the first metal specimen, secondary particles formed by agglomerating first metal primary particles with an average particle size (D50) of 5 μm or less may be grown.

In the forming of the second metal hydroxide layer on the surface of the first metal specimen on which the micro-sized first metal particles are grown, electrodeposition may be performed by immersing the first metal specimen on which the micro-sized first metal particles are grown in an alkaline solution in which a second metal salt is dissolve.

In the forming of the second metal hydroxide layer on the surface of the first metal specimen on which the micro-sized first metal particles are grown, one or more second metal hydroxide islands may be formed on the surface of the first metal specimen.

In the forming of the second metal hydroxide layer on the surface of the first metal specimen on which the micro-sized first metal particles are grown, a second metal hydroxide coating layer may be formed on an area in a range of 5% to 20% of the surface of the first metal specimen.

In the forming of the second metal hydroxide layer on the surface of the first metal specimen on which the micro-sized first metal particles are grown, a second metal hydroxide coating layer having an average thickness in the range of 0.1 μm to 1 μm may be formed.

The first metal and the second metal may be one or more selected from the group consisting of nickel (Ni), copper (Cu), vanadium (V), cobalt (Co), molybdenum (Mo), and tungsten (W), the first metal may include nickel (Ni), and the second metal may include copper (Cu).

Yet another embodiment of the present invention provides a process for producing for FDCA, through a process using a catalyst that minimizes side reactions due to fructose by injecting a crude HMF solution containing residual fructose into an electrochemical reactor including a catalyst electrode, wherein the catalyst electrode may be applied as the catalyst.

FDCA may be produced by applying a potential in a range of 1.30 V_{RHE} to 1.70 V_{RHE} to the catalyst electrode.

The catalyst electrode according to an embodiment of the present invention and a process for production of FDCA using the same has the advantage in that FDCA using crude HMF may be produced.

The catalyst electrode according to an embodiment of the present invention and a process for production of FDCA using the same has the advantage in that a production cost of FDCA may be reduced.

### BRIEF DESCRIPTION OF THE DRAWINGS

FIG. 1 is a schematic diagram of a catalyst electrode for production of FDCA according to an embodiment of the present invention.

FIG. 2 illustrates results of scanning electron microscopy (SEM) analysis of the catalyst electrode according to Example 1 of the present invention.

FIG. 3 illustrates results of scanning electron microscopy-energy dispersive X-ray spectroscopy (SEM-EDS) analysis of the catalyst electrode according to Example 1 of the present invention.

FIG. 4 illustrates results of SEM-EDS analysis of the catalyst electrode according to Example 2 of the present invention.

FIG. 5 illustrates results of cross-sectional SEM analysis of the catalyst electrode according to Example 2 of the present invention.

FIG. 6 illustrates results of X-ray absorption spectroscopy (XAS) analysis of the catalyst electrode according to Example 1 of the present invention.

FIG. 7A and FIG. 7B illustrate results of analyzing properties of a HMF oxidation reaction according to Experimental Example 1 of the present invention as a current-voltage curve graph of a constant potential method and FDCA yield and faraday efficiency.

FIG. 8A, FIG. 8B and FIG. 8C illustrate results of analyzing properties of a crude HMF oxidation reaction according to Experimental Example 2 of the present invention as a current-voltage curve graph of a constant potential method and FDCA yield and faraday efficiency.

FIG. 9A, FIG. 9B and FIG. 9C illustrate results of analyzing properties of a crude HMF oxidation reaction according to Experimental Example 3 of the present invention as a current-voltage curve graph of a constant potential method and FDCA yield and faraday efficiency.

FIG. 10A and FIG. 10B illustrate results of calculating a FDCA production rate according to Experimental Example 3 of the present invention.

FIG. 11 illustrates results of calculating a formic acid production rate according to Experimental Example 3 of the present invention.

FIG. 12 illustrates a photograph of a FDCA solution produced by applying a catalyst electrode according to Experimental Example 3 of the present invention.

FIG. 13A and FIG. 13B illustrate results of nuclear magnetic resonance (NMR) analysis of FDCA produced by applying the catalyst electrodes according to Experimental Examples 1 and 3 of the present invention.

### DETAILED DESCRIPTION OF THE EMBODIMENTS

Technical terms used herein are to mention only a specific embodiment, and are not to limit the present invention. Singular forms used herein include plural forms as long as phrases do not clearly indicate an opposite meaning. As used in the specification, the term “including” concretely indicates specific properties, regions, integer numbers, steps, operations, elements, and/or components, and is not intended to exclude existence or addition of other properties, regions, integer numbers, steps, operations, elements and/or components.

Unless otherwise defined, all terms used herein, including technical and scientific terms, have the same meaning as commonly understood by one of ordinary skill in the art to which the present invention belongs. Terms defined in a generally used dictionary are additionally interpreted as having the meaning matched to the related art document and the currently disclosed contents and are not interpreted as ideal or formal meaning unless defined.

Terms such as first, second, and third are used to describe, but are not limited to, various parts, components, regions, layers, and/or sections. These terms are used only to distinguish one part, component, area, layer, or section from another part, component, area, layer, or section. Thus, a first component, part, region, layer, or section described below may be referred to as a second component, part, region, layer, or section without departing from the scope of the present invention.

Hereinafter, embodiments of the present invention will be described in detail. However, it is to be understood that this is presented as an example, and the present invention is not limited thereby and is only defined by the scope of the following claims.

One embodiment of the present invention may provide a catalyst electrode for production of FDCA.

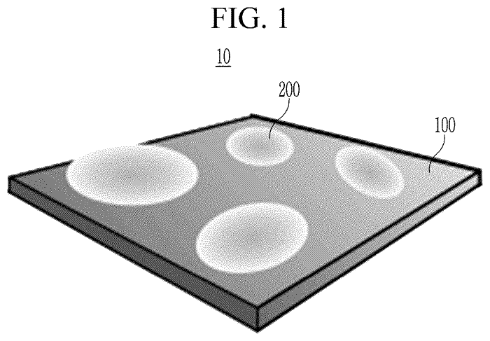

FIG. 1 is a schematic diagram of a catalyst electrode for production of FDCA according to an embodiment of the present invention.

Referring to FIG. 1, a catalyst electrode 10 for production of FDCA according to one embodiment of the present invention may include a first metal substrate 100 and a second metal hydroxide layer 200.

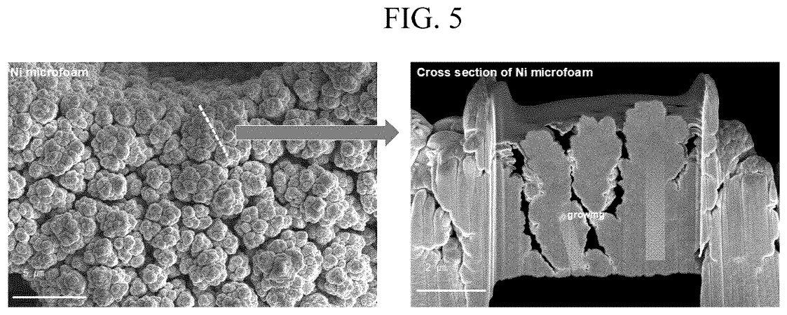

Referring to FIG. 5, the first metal substrate 100 of the catalyst electrode of the present invention may include one or more micro-sized first metal particles on the surface thereof, and the micro-sized first metal particles may include one or more secondary particles formed by agglomerating first metal primary particles with an average particle size (D50) of 5 μm or less, and specifically, may include one or more secondary particles formed by agglomerating first metal primary particles with an average particle size (D50) of 1 μm to 5 μm.

The first metal and the second metal may be different transition metals, and specifically may be one or more selected from the group consisting of nickel (Ni), copper (Cu), vanadium (V) cobalt (Co) molybdenum (Mo), and tungsten (W), and more specifically may be nickel (Ni).

The first metal substrate 100 may include one or more types selected from a metal foam and a metal foil. In the case of the metal foam, pores may be formed on the surface, and specifically, the metal foam may include surface pores having an average diameter in a range of 1 μm to 300 μm.

The second metal hydroxide layer 200 may be dispersed and located on the surface of the first metal substrate 100 in an island shape, and may be uniformly dispersed and located.

On the other hand, the second metal hydroxide layer 200 may be located on the surface of the micro-sized first metal particles.

The islands of the second metal hydroxide layer 200 may have an average diameter in the range of 1 μm to 300 μm relative to the surface of the first metal substrate 100. The average diameter may be an average diameter of the shortest and longest diameters of the second metal hydroxide layer 200, which forms one intact surface without fracture.

Meanwhile, the second metal hydroxide layer 200 may be applied to a portion of the surface of the first metal substrate 100, and specifically may be located on an area in a range of 1% to 50% of the surface of the first metal substrate 100, and more specifically may be located on an area in a range of 5% to 20%. When the second metal hydroxide layer 200 is located in the above range, there is an advantage in that the production efficiency and purity of FDCA are improved.

The second metal hydroxide layer 200 may have an average thickness in the range of 0.1 μm to 1 μm. If the average thickness of the second metal hydroxide layer 200 is in the above range, there is an advantage that FDCA may be produced efficiently while reducing the production cost of the catalyst for production of FDCA.

Another embodiment of the present invention may provide a method for preparation of a catalyst electrode for production of FDCA.

A method for preparation of a catalyst electrode for production of FDCA according to another embodiment of the present invention may include: pretreating a first metal specimen; growing micro-sized first metal particles on a surface of the pretreated first metal specimen; and forming a second metal hydroxide layer on the surface of the first metal specimen on which the micro-sized first metal particles are grown on the surface.

Here, the first metal and second metal may be different transition metals, and specifically, may be one or more selected from the group consisting of nickel (Ni), copper (Cu), vanadium (V) cobalt (Co) molybdenum (Mo), and tungsten (W). Further, the first metal may be nickel (Ni) and the second metal may be copper (Cu).

The pretreating of the first metal specimen may include surface treating the first metal specimen in an aqueous acid solution, and performing treatment by immersing the surface-treated first metal specimen in a first metal salt solution.

In the surface treating of the first metal specimen in an aqueous acid solution, the acid solution may be one or more acid solutions selected from sulfuric acid, nitric acid, hydrochloric acid, and phosphoric acid. The first metal specimen may be immersed in the acid solution for a predetermined time.

In the growing of the micro-sized first metal particles on the surface of the pretreated first metal specimen, electrodeposition may be performed in a first constant current method by immersing the first metal specimen in the first metal salt solution. The first metal salt may be one or more selected from sulfate, nitrate, hydrochloride, and phosphate. The first metal porous structure may be formed by immersing the first metal specimen in the first metal salt solution and growing micro-particles on the surface of the first metal specimen in a constant current method at a current in a range of −0.1 A to −2 A, and particularly at a current of −0.5 A per unit area (cm²) for 3 minutes. The first metal frame may be formed by agglomerating first metal primary particles with an average particle size (D50) of 5 μm or less, and specifically 1 μm to 5 μm.

In the forming of the second metal hydroxide layer on the surface of the first metal specimen on which the micro-sized first metal particles are grown, by immersing a first metal specimen on which micro-sized first metal particles have grown on the surface in an alkaline solution in which a second metal salt is dissolved, a current may be applied using a constant voltage method to form a second metal hydroxide on the surface of the first metal specimen, and specifically, one or more islands of second metal hydroxide may be formed. The second metal hydroxide may form a second metal hydroxide coating layer on an area in the range of 1% to 50% of the surface of the first metal specimen, and specifically, on an area in the range of 5% to 20% of the surface of the first metal specimen. In addition, the second metal hydroxide may have an average thickness in the range of 0.1 μm to 1 μm.

Another embodiment of the present invention provides a process for production of FCDA using the catalyst electrode for production of FCDA. In a HMF production process, a crude HMF solution containing residual fructose and other by-products is introduced into an electrochemical reactor including the catalyst electrode, and a potential in the range of 1.30 V_{RHE} to 1.70 V_{RHE} is applied to the catalyst electrode to produce high purity FDCA. Specifically, by using a crude HMF solution as a raw material, rather than a highly purified HMF solution, in which the purification process of residual fructose and impurities is omitted, high-purity FDCA may be produced more economically.

Hereinafter, embodiments of the present invention will be described in detail. However, it is to be understood that this is presented as an example, and the present invention is not limited thereby and is only defined by the scope of the following claims.

### Example 1 (Ni Foam/Cu OH)₂

A nickel foam used as a metal support layer was immersed in a 0.5 M hydrochloric acid (HCl) aqueous solution to clean a surface of the nickel foam. The surface-cleaned nickel foam was immersed in a 0.1 M NaOH aqueous solution (pH 13) containing 0.0158 mM (1 ppm) CuCl₂ dissolved, and copper hydroxide was formed on the surface of the nickel foam through an oxidation reaction at an applied voltage of 1.5 V_{RHE} for 1 min using a constant voltage method. Specifically, in an electrolyte in an alkaline environment, copper ions are combined with four hydroxyl groups and dissolved as Cu(OH)₄²⁻. When the nickel foam is immersed in an electrolyte in which trace amounts of Cu(OH)₄²⁻ are dissolved with an anionic nature and an oxidizing voltage is applied, the Cu(OH)₄²⁻ is oxidized and deposited as Cu(OH)₂ on a surface of a nickel electrode to form a catalyst electrode. The size and thickness of the deposited Cu(OH)₂ may be controlled by adjusting the CuCl₂ concentration and the oxidation reaction time.

### Comparative Example 1 (Ni Foam)

The catalyst electrode was prepared using a nickel foam.

### Comparative Example 2 (Cu Foam)

The catalyst electrode was prepared using a copper foam.

### Example 2 (Ni Foam/Ni Microfoam (MF)/Cu OH)₂

A nickel foam used as a metal support layer was immersed in a 0.5 M hydrochloric acid (HCl) aqueous solution to clean a surface of the nickel foam. Immersion was performed in an aqueous solution containing a mixture of 0.1 M NiCl₂ and 1 M NH₄Cl, and a nickel microfoam (MF) structure with a porous frame consisting of micrometer-sized particles was formed on the surface of the nickel foam through a reduction reaction at an applied current of 0.5 A/cm² for 3 min using a constant current method. The prepared nickel foam was immersed in a 0.1 M NaOH aqueous solution (pH 13) containing dissolved 0.0158 mM (1 ppm) CuCl₂, and copper hydroxide was formed on the surface of the nickel foam through an oxidation reaction at an applied voltage of 1.5 V_{RHE} for 1 min using a constant voltage method.

### Comparative Example 2-1 (Ni Foam/Ni MF)

A nickel foam used as a metal support layer was immersed in a 0.5 M hydrochloric acid (HCl) aqueous solution to clean the surface of the nickel foam. Immersion was performed in an aqueous solution containing a mixture of 0.1 M NiCl₂ and 1 M NH₄Cl, and a nickel MF structure with a porous frame consisting of micrometer-sized particles was formed on the surface of the nickel foam through a reduction reaction at an applied current of 0.5 A/cm² for 3 min using a constant current method.

### Comparative Example 2-2 (Cu Foam/Cu MF)

A copper foam used as a metal support layer was immersed in a 0.1 M sulfuric acid (H₂SO₄) aqueous solution to clean the surface of the copper foam. The surface-treated copper foam was immersed in an aqueous solution mixed with 0.1 M CuSO₄, 0.3 M H₂SO₄, and 0.1 M K₂SO₄, and a reduction reaction was carried out at −0.6 V relative to a relative electrode using a constant voltage method, and a charge amount of 30 C was applied to form copper seeds. A second constant voltage method was carried out to form a micro-sized dendrite structure on the formed copper seeds to create a porous structure. In the same electrodeposition solution, a charge of 30 C was applied to form a copper foam structure with a porous frame by conducting a reduction reaction at −2V relative to the relative electrode.

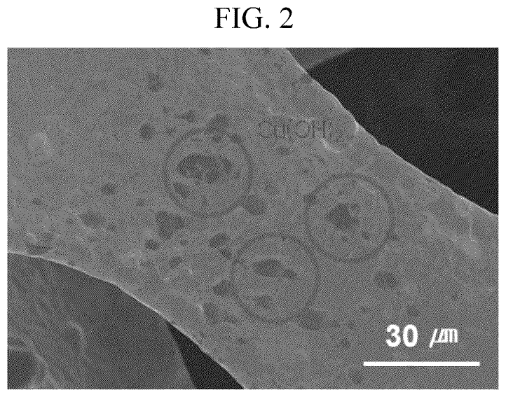

FIG. 2 illustrates results of SEM analysis of the catalyst electrode according to Example 1 of the present invention.

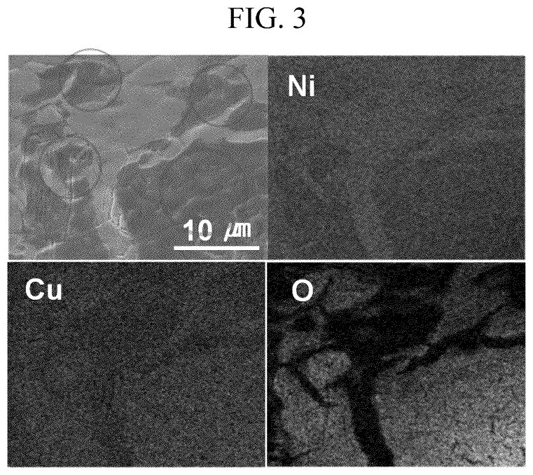

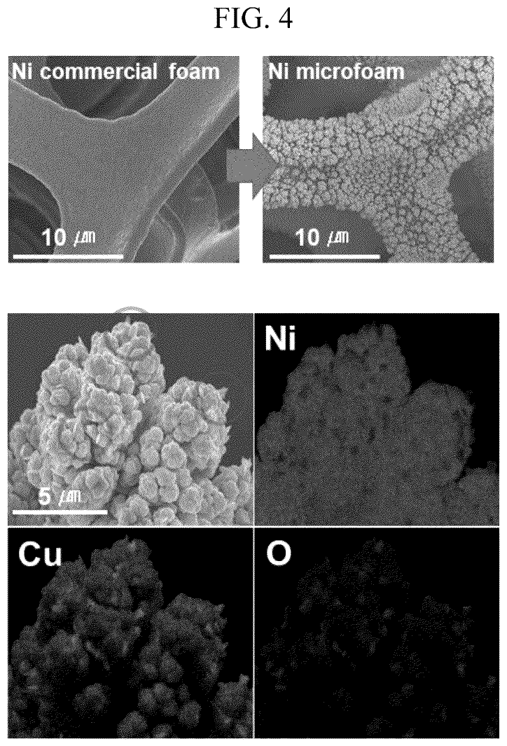

FIGS. 3 and 4 illustrate results of SEM-EDS analysis of the catalyst electrodes according to Examples 1 and 2 of the present invention.

In the present invention, the SEM analysis was performed at an accelerated voltage of 15 KV using a Hitachi's Regulus 8230 instrument, and the SEM-EDS analysis was performed at an accelerated voltage of 15 kV using an EDS attachment attached to the Hitachi's Regulus 8230.

Referring to FIGS. 2 and 3, it can be confirmed that copper hydroxide with a diameter of less than 20 μm was deposited in an island shape on the surface of the nickel foam. In addition, the copper and oxygen regions detected by mapping are identical, indicating that they are present as copper hydroxide rather than copper metal.

Referring to FIG. 4, it can be confirmed that copper elements were deposited on the surface of the nickel MF as a whole, and copper hydroxide of 1 μm or less was deposited in the form of particles. In addition, the copper and oxygen regions detected by mapping are identical, indicating that they are present as copper hydroxide rather than copper metal.

The metal and oxygen elemental contents according to each Examples are shown in Table 1 below.

TABLE 1

| Element | Example 1 | Example 2 |
|---|---|---|
| Ni (at %) | 71.11 | 87.54 |
| Cu (at %) | 1.28 | 6.63 |
| O (at %) | 27.60 | 5.83 |

FIG. 5 illustrates results of cross-sectional SEM analysis of the catalyst electrode according to Example 2 of the present invention.

Referring to FIG. 5, a cross-section of a nickel MF catalyst electrode grown by agglomeration of nickel micro-primary particles with a diameter of 5 μm or less on a nickel foam surface can be seen.

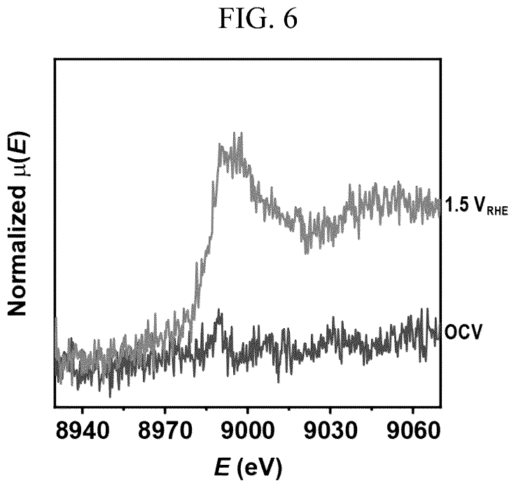

FIG. 6 illustrates results of XAS analysis of the catalyst electrode according to Example 1 of the present invention.

Referring to FIG. 6, the catalyst electrode was immersed in an electrolyte in which Cu(OH)₄²⁻ ions are dissolved in a real-time X-ray analysis environment, and Cu K-edge absorption was observed from open circuit condition (OCV) to the applied potential of 1.5 V_{RHE}. The Cu element was not detected at the OCV condition, but the Cu element was detected at the 1.5 V_{RHE}, demonstrating that copper hydroxide is deposited on the nickel surface through the oxidation reaction even in the electrolyte in which several ppm of copper ions are dissolved.

### FDCA Production Experiment Using Catalyst Electrode

In the present invention, an FDCA production reactor may be used, the reactor including an inlet to which a crude HMF solution is introduced, a catalytic electrode, and an outlet for discharging FDCA produced after an oxidation reaction of HMF performed at the catalyst electrode. Inside the FDCA reactor, a catalytic electrode and a cathode provided in a form facing the catalytic electrode may be provided. The catalytic electrode may function as an anode within the reactor. An electrochemical reaction in which HMF, an intermediate product, is converted to FDCA may be performed by the catalyst electrode and the cathode.

A conductive separator may be further provided inside the reactor. A conductive separator separates the catalyst electrode and cathode provided inside the reactor. Thus, on a catalytic electrode side of the reactor separated by the conductive separator, an electrochemical conversion reaction of HMF to FDCA may be carried out, and on a cathode side of the reactor, a reaction of water conversion into hydrogen may be carried out.

In the FDCA production reactor, FDCA may be produced by applying a potential of 1.30 V_{RHE} to 1.70 V_{RHE} to the catalyst electrode.

Meanwhile, the residual fructose in the crude HMF solution may be converted to formic acid through an oxidation reaction as shown in Scheme 1 below.

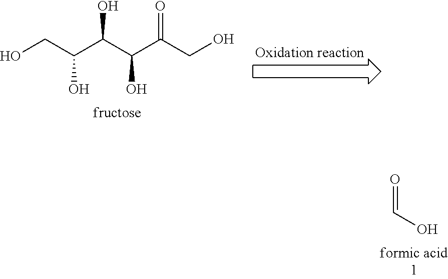 [SMILES: O=C(CO)[C@@H](O)[C@H](O)[C@H](O)CO ; O=CO]

### Experimental Example 1

An FDCA production experiment was performed using pure HMF as a raw material and an FDCA reactor including catalytic electrodes according to Example 1 and Comparative Examples 1-1 and 1-2.

### Experimental Example 2

An FDCA production experiment was performed using an actual crude HMF solution as a raw material and an FDCA reactor including a catalyst electrode according to Example 1 and Comparative Examples 1-1 and 1-2.

### Experimental Example 3

An FDCA production experiment was performed using an actual crude HMF solution as a raw material and an FDCA reactor including a catalyst electrode according to Example 2 and Comparative Examples 2-1 and 2-2.

A composition ratio and a feed flow rate of a mixture of fructose and HMF in the actual crude HMF solution are shown in Table 2 below.

TABLE 2

| Chemicals | wt/wt in H₂O (%) |
|---|---|
| HMF | 40.86 |
| Fructose + Glucose | 2.025 |
| Furyl-hydroxymethyl-ketone | 1.62 |
| Furfural | 0.405 |
| Formaldehyde | 0.045 |

The electrochemical conversion reactions according to Experimental Example 1, Experimental Example 2, and Experimental Example 3 were analyzed.

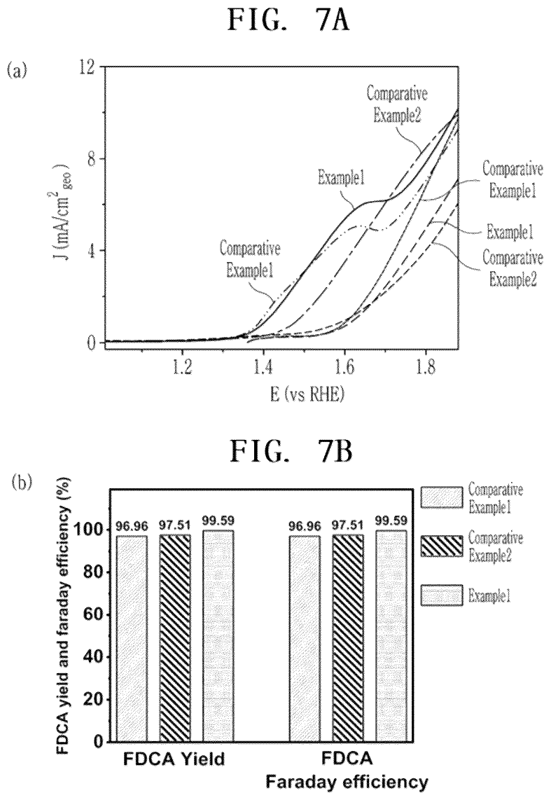

FIG. 7A and FIG. 7B illustrate a current-voltage curve of a linear sweep voltammetry (LSV) method, which characterized the HMF oxidation reaction occurring at the catalytic electrode according to Experimental Example 1 of the present invention, and the yield and electron usage efficiency of FDCA produced by flowing the theoretical amount of charge required for HMF oxidation applying a constant potential of 1.5 V_{RHE}. In Example 1 where pure HMF was used as the raw material, since side reactions other than the HMF oxidation reaction rarely occur, 95% or more of a theoretical charge flowed was all used for the FDCA production reaction.

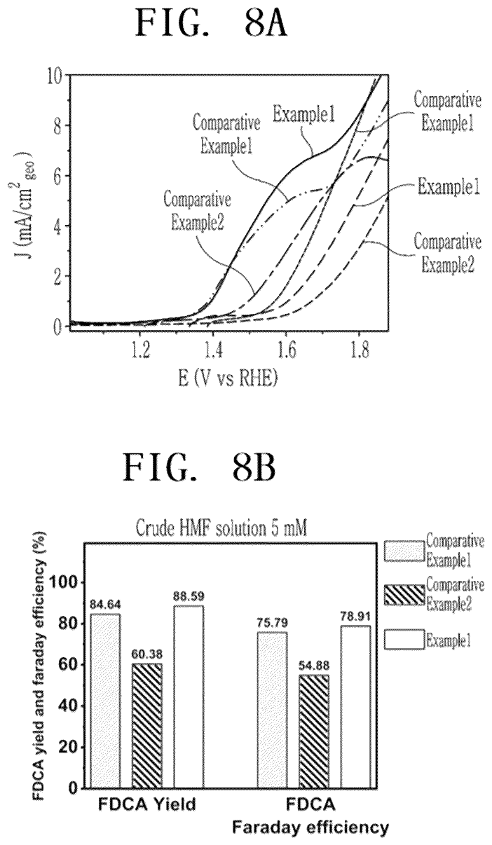

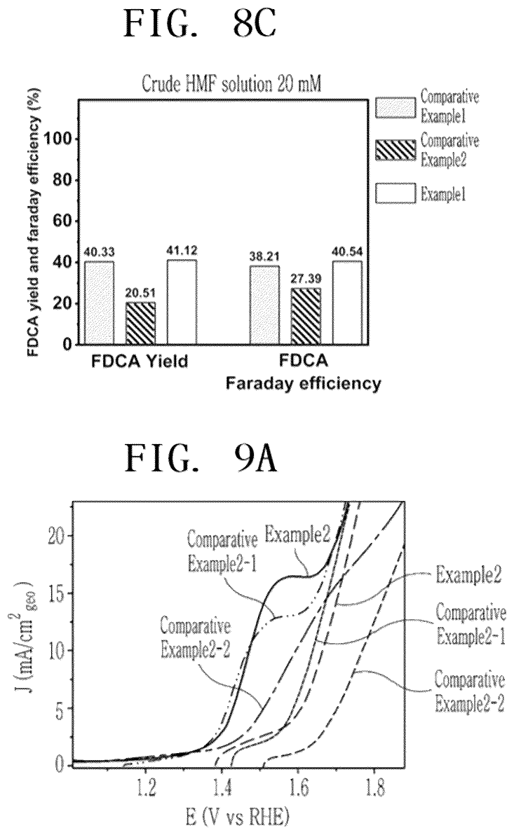

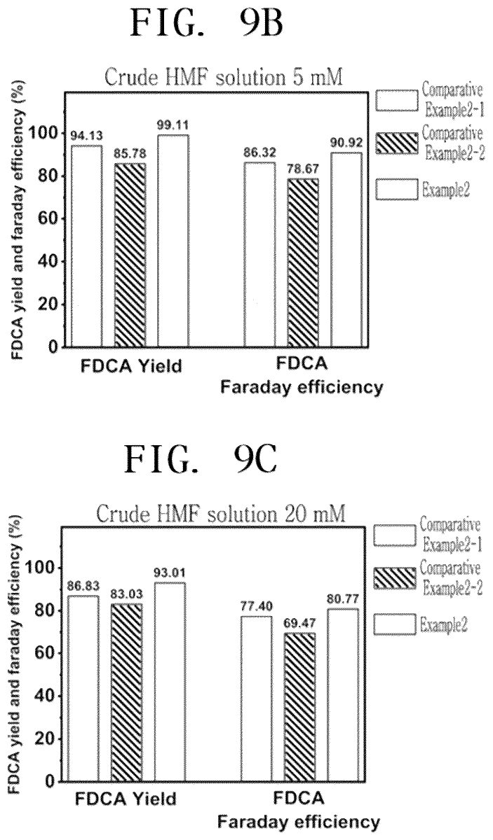

FIG. 8A to FIG. 8C and FIG. 9A to FIG. 9B illustrate current-voltage curves of a linear sweep voltammetry (LSV) method, which characterized the oxidation reaction of a crude HMF solution occurring at the catalytic electrode according to Experiment 2 and Experiment 3 of the present invention, and the yield and electron usage efficiency of FDCA produced by flowing a theoretical charge required for HMF oxidation by applying a constant potential of 1.5 V_{RHE}.

Referring to FIG. 8A to FIG. 8C and FIG. 9A to FIG. 9B, it can be confirmed that nickel, which is favorable for HMF oxidation, initiates the overall reaction faster than copper. Nickel in which copper hydroxide (Ni/Cu(OH)₂) is deposited starts to form current values at similar voltages compared to nickel, but an increasing reaction current value around 1.5 V_{RHE} was found to be about 1.5 times higher. In addition, FDCA yield and electron usage efficiency were higher in all concentrations of crude HMF solution.

It can be confirmed that the reason for this performance on the Ni/Cu(OH)₂ is due to the different superior performances of nickel and copper in oxidizing HMF and residual fructose, and the complementary reaction of these two metals on the surface resulted in more oxidation reactions.

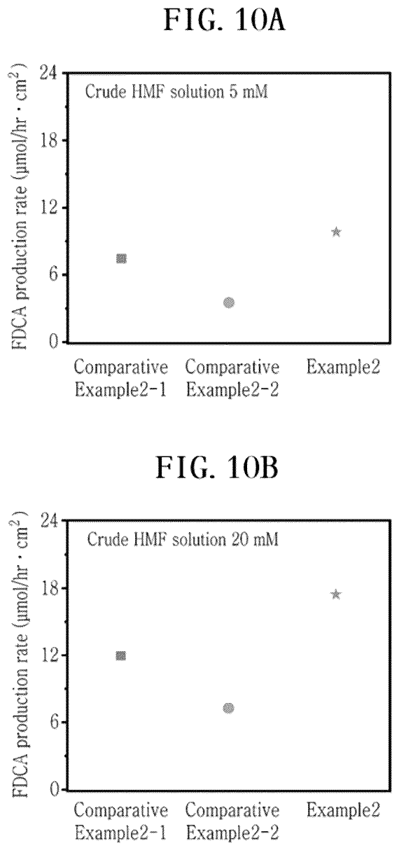

FIG. 10A to FIG. 10B illustrate an FDCA production rate according to Experimental Example 3 of the present invention. Referring to FIG. 10A to FIG. 10B, it can be confirmed that in Experiment 3 using the crude HMF as a raw material, the highest FDCA yield and electron usage efficiency, as well as the fastest FDCA production rate, were obtained when a Ni/Cu(OH)₂ was deposited was used.

The composition analysis results of the final products according to Experimental Example 1, Experimental Example 2, and Experimental Example 3 are summarized in Table 3 below.

TABLE 3

| | | | | End |
|---|---|---|---|---|
| | | FDCA | FDCA electron usage | time |
| Electrode | Reactant | yield (%) | efficiency (FE) (%) | (hr) |
| Ni | HMF 5 mM | 97.92 | 97.59 | 5.5 |
| (Comparative | Crude HMF | 84.64 | 75.79 | 36.0 |
| Example1-1) | 5 mM | | | |
| | Crude HMF | 40.33 | 38.21 | 36.0 |
| | 20 mM | | | |
| Ni microfoam | HMF 5 mM | 97.15 | 92.95 | 4.8 |
| (Comparative | Crude HMF | 94.13 | 86.32 | 10.8 |
| Example2-1) | 5 mM | | | |
| | Crude HMF | 86.83 | 77.40 | 22.8 |
| | 20 mM | | | |
| Cu | HMF 5 mM | 97.41 | 96.51 | 8.7 |
| (Comparative | Crude HMF | 60.38 | 54.88 | 36.0 |
| Example1-2) | 5 mM | | | |
| | Crude HMF | 20.51 | 27.39 | 36.0 |
| | 20 mM | | | |
| Cu microfoam | HMF 5 mM | 97.54 | 96.25 | 7.8 |
| (Comparative | Crude HMF | 89.21 | 81.79 | 20.1 |
| Example2-2) | 5 mM | | | |
| | Crude HMF | 83.03 | 69.47 | 32.9 |
| | 20 mM | | | |
| Ni/Cu(OH)2 | HMF 5 mM | 99.60 | 98.87 | 4.6 |
| (Example1) | Crude HMF | 88.59 | 78.91 | 36.0 |
| | 5 mM | | | |
| | Crude HMF | 41.12 | 40.54 | 36.0 |
| | 20 mM | | | |
| Ni microfoam/ | HMF 5 mM | 99.72 | 99.53 | 4.2 |
| Cu(OH)2 | Crude HMF | 99.11 | 90.92 | 8.4 |
| (Example2) | 5 mM | | | |
| | Crude HMF | 93.01 | 80.77 | 16.8 |
| | 20 mM | | | |

Referring to Table 3, the FDCA production performances according to Experimental Example 1, Experimental Example 2, and Experimental Example 3 was compared using nickel, copper, and Ni/Cu(OH) 2 electrodes.

From the results of the three FDCA production experiments, it can be confirmed that the Ni/Cu(OH)₂ is deposited according to the present invention exhibited superior FDCA production performance compared to the comparative examples from the three comprehensive aspects of FDCA yield, electron usage efficiency, and production rate.

In addition, it was confirmed that when the crude HMF was used as a raw material, the FDCA yield was 80% or more in both Example 1 and Example 2 at a low concentration of 5 mM, but when the concentration was increased to 20 mM, the FDCA yield of Example 1 was reduced to 50% or less, and the FDCA yield of Example 2 was maintained at 90% or more. Therefore, it can be confirmed that the best FDCA yield is obtained when the nickel microfoam catalyst electrode in which copper hydroxide was deposited (Ni MF/Cu(OH)₂) according to the present invention was used.

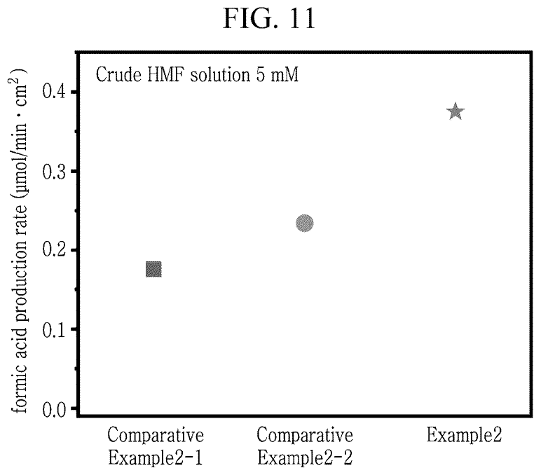

FIG. 11 illustrates a formic acid production rate according to Experimental Example 3 of the present invention. Referring to FIG. 11, in Experimental Example 3, in addition to a reaction in which HMF was converted to FDCA, a reaction in which residual fructose is converted to formic acid may occur. In order to minimize side reactions due to fructose, the conversion reaction of fructose to formic acid needs to occur quickly. In Example 3, it was confirmed that the FDCA yield was the best because formic acid was produced the fastest.

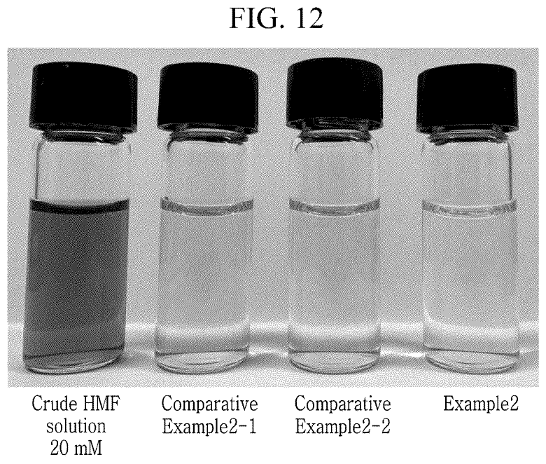

FIG. 12 illustrates a photograph of a FDCA solution produced by applying the catalyst electrode according to Experimental Example 3 of the present invention.

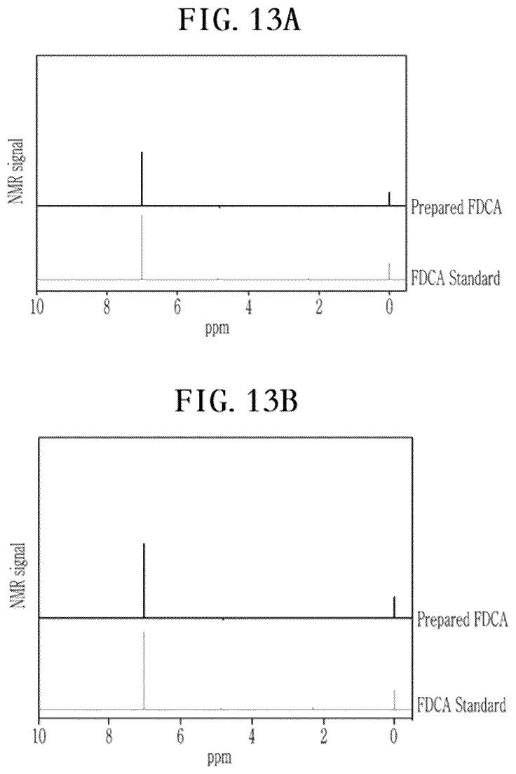

FIG. 13A to FIG. 13B illustrate results of NMR analysis of FDCA produced by applying the catalyst electrodes according to Experimental Examples 1 and 3 of the present invention.

Specifically, FIG. 13A illustrates the analysis results according to Example 1 in Experimental Example 1, and FIG. 13B illustrates analysis results according to Example 2 in Experimental Example 3.

Referring to FIG. 13A to FIG. 13B, it can be confirmed that when Example 2 according to the present invention was used, the FDCA produced according to Experiment 1 and Experiment 3 exhibited the same 1H NMR signal as the reference FDCA, no signal caused by a substance other than FDCA was identified, and the purity was 99.9% or more because the signal has an area close to 99.9% of a measured FDCA concentration.

The present invention is not limited to the above embodiments, but May be prepared in a variety of different forms, and one of ordinary skilled one having ordinary skill in the art to which the invention belongs will understand that the present invention may be practiced in other specific forms without altering the technical idea or essential features of the present invention. Therefore, it is to be understood that the embodiments described above are illustrative rather than being restrictive in all aspects.

What is claimed is:

1. A catalyst electrode for production of 2,5-furandicarboxylic acid, comprising:
a first metal substrate; and
a second metal hydroxide coating layer located on a surface of the first metal substrate,
wherein the first metal substrate includes one or more micro-sized first metal particles on the surface thereof, and
the second metal hydroxide coating layer is located on a surface of the first metal particles.

2. The catalyst electrode for production of 2,5-furandicarboxylic acid of claim 1, wherein
the micro-sized first metal particles are secondary particles formed by agglomerating first metal primary particles with an average particle size (D50) of 5 μm or less,

3. The catalyst electrode for production of 2,5-furandicarboxylic acid of claim 1, wherein
the second metal hydroxide is dispersed and located on the surface of the first metal substrate in an island shape.

4. The catalyst electrode for production of 2,5-furandicarboxylic acid of claim 1, wherein
the second metal hydroxide is located on 5% to 20% of a surface area of the first metal substrate.

5. The catalyst electrode for production of 2,5-furandicarboxylic acid of claim 1, wherein
the second metal hydroxide has an average thickness in a range of 0.1 μm to 1 μm.

6. The catalyst electrode for production of 2,5-furandicarboxylic acid of claim 1, wherein
the first metal substrate includes one or more types selected from a nickel foam and a nickel foil.

7. The catalyst electrode for production of 2,5-furandicarboxylic acid of claim 1, wherein
the second metal hydroxide coating layer is a copper hydroxide coating layer.

8. A method for preparation of a catalyst electrode for production of 2,5-furandicarboxylic acid, comprising:
growing micro-sized first metal particles on a surface of a first metal specimen; and
forming a second metal hydroxide layer on the surface of the first metal specimen on which the micro-sized first metal particles are grown,
wherein the first metal and the second metal are different transition metals.

9. The method for preparation of a catalyst electrode for production of 2,5-furandicarboxylic acid of claim 8, wherein
in the growing of the micro-sized first metal particles on the surface of the first metal specimen,
electrodeposition is performed in a constant current method by immersing the first metal specimen in a first metal salt solution.

10. The method for preparation of a catalyst electrode for production of 2,5-furandicarboxylic acid of claim 8, wherein
in the growing of the micro-sized first metal particles on the surface of the first metal specimen,
secondary particles formed by agglomerating first metal primary particles with an average particle size (D50) of 5 μm or less are grown.

11. The method for preparation of a catalyst electrode for production of 2,5-furandicarboxylic acid of claim 8, wherein
in the forming of the second metal hydroxide layer on the surface of the first metal specimen on which the micro-sized first metal particles are grown,
electrodeposition is performed by immersing the first metal specimen on which the micro-sized first metal particles are grown in an alkaline solution in which a second metal salt is dissolved.

12. The method for preparation of a catalyst electrode for production of 2,5-furandicarboxylic acid of claim 8, wherein
in the forming of the second metal hydroxide layer on the surface of the first metal specimen on which the micro-sized first metal particles are grown,
one or more second metal hydroxide islands are formed on the surface of the first metal specimen.

13. The method for preparation of a catalyst electrode for production of 2,5-furandicarboxylic acid of claim 8, wherein
in the forming of the second metal hydroxide layer on the surface of the first metal specimen on which the micro-sized first metal particles are grown,
a second metal hydroxide coating layer is formed on an area in a range of 5% to 20% of the surface of the first metal specimen.

14. The method for preparation of a catalyst electrode for production of 2,5-furandicarboxylic acid of claim 8, wherein
in the forming of the second metal hydroxide layer on the surface of the first metal specimen on which the micro-sized first metal particles are grown,
a second metal hydroxide coating layer having an average thickness in the range of 0.1 μm to 1 μm is formed.

15. The method for preparation of a catalyst electrode for production of 2,5-furandicarboxylic acid of claim 8, wherein
the first metal and the second metal are one or more selected from the group consisting of nickel (Ni), copper (Cu), vanadium (V), cobalt (Co), molybdenum (Mo), and tungsten (W).

16. The method for preparation of a catalyst electrode for production of 2,5-furandicarboxylic acid of claim 15, wherein
the first metal includes nickel (Ni).

17. The method for preparation of a catalyst electrode for production of 2,5-furandicarboxylic acid of claim 15, wherein
the second metal includes copper (Cu).

18. A process for producing 2,5-furandicarboxylic acid with high purity, through a process using a catalyst that minimizes side reactions due to fructose by injecting a crude HMF solution containing residual fructose into an electrochemical reactor including a catalyst electrode,
wherein the catalyst electrode of claim 1 is applied as the catalyst.

19. The process for producing 2,5-furandicarboxylic acid of claim 18, wherein
high-purity 2,5-furandicarboxylic acid is produced by applying a potential in a range of 1.30 V_{RHE} to 1.70 V_{RHE} to the catalyst electrode.
